(19) United States
(12) Patent Application Publication
(10) Pub. No.: US 2023/0106488 A1
(43) Pub. Date: Apr. 6, 2023

(54) METHOD FOR DETERMINING SECRETED LEVELS OF FUCOSYLATED HUMAN MILK OLIGOSACCHARIDES

(71) Applicant: SOCIETE DES PRODUITS NESTLE S.A., Vevey (CH)

(72) Inventors: ARISTEA BINIA, Fribourg (CH); GREGORY LEFEBVRE, Vuillierens (CH)

(21) Appl. No.: 17/907,137

(22) PCT Filed: Mar. 26, 2021
(86) PCT No.: PCT/EP2021/057923

§ 371 Date: Sep. 23, 2022

(30) Foreign Application Priority Data
Mar. 27, 2020 (EP) 20166465.3

(51) Int. Cl. C12Q 1/6827; C12Q 1/6858

(52) U.S. Cl. CPC C12Q 1/6827; C12Q 1/6858; C12Q 2600/124

(57) ABSTRACT

The present invention concerns a polygenic score and a method embedding such polygenic score for predicting levels of fucosylated human milk oligosaccharides, for example alpha-1,2-fucosylated human milk oligosaccharides secreted by a human subject during lactation.

### FIELD OF THE INVENTION

The present invention concerns a polygenic score and a method for predicting levels of fucosylated human milk oligosaccharides secreted by a human subject during lactation embedding such polygenic score. The present invention also relates to a method to provide nutritional recommendations for supplementation of fucosylated human milk oligosaccharides in the diet of an infant and/or a young child.

In particular, the present invention concerns a polygenic score and a method for predicting levels of alpha-1,2-fucosylated human milk oligosaccharides secreted by a human subject during lactation embedding such polygenic score. The present invention also relates to a method to provide nutritional recommendations for supplementation of alpha-1,2-fucosylated human milk oligosaccharides in the diet of an infant and/or a young child.

The present invention also concerns a polygenic score and a method for predicting levels of alpha-1,3/4-fucosylated human milk oligosaccharides secreted by a human subject during lactation embedding such polygenic score. The present invention also relates to a method to provide nutritional recommendations for supplementation of alpha-1,3/4-fucosylated human milk oligosaccharides in the diet of an infant and/or a young child.

### BACKGROUND OF THE INVENTION

Human milk oligosaccharide (HMO) expression in human breast milk is highly variable and depends on genetics, time of lactation and potentially other unknown factors. Especially, it has been observed that certain fucosylated HMOs, like alpha-1,2-fucosylated HMOs, for example 2′-fucosylactose (2′FL), can vary substantially among lactating mothers.

Secretors (FUT2 enzyme active) express alpha-1,2-fucosylated HMOs (2′FL and other related HMO) and non-secretors (FUT2 enzyme inactive) have almost zero levels of such HMOs. This is known to be controlled by genetic factors. However, even within Secretors (FUT2 enzyme active), high variability exists that cannot be fully explained by FUT2 enzyme activity alone. Knowing the expressed levels of alpha-1,2-fucosylated HMOs in certain human breast milk is of relevance to understand whether such HMOs should be supplemented to the infant receiving that milk and if so at which amounts.

Mothers who are expressors (with FUT3 enzyme active) express alpha-1,3/4-fucosylated HMOs (3FL and other related HMOs) and mothers who are non-expressors (FUT3 enzyme inactive) have almost zero or very low levels of such HMOs. This is known to be controlled by genetic factors. However, even within non-expressors (FUT3 enzyme active), high variability exists that cannot be fully explained by FUT3 enzyme activity alone. Knowing the expressed levels of alpha-1,3/4-fucosylated HMOs in certain human breast milk is of relevance to understand whether such HMOs should be supplemented to the infant receiving that milk and if so at which amounts.

Existing solutions to assess such levels include measuring the amounts of alpha-1,2-fucosylated HMOs or of of alpha-1,3/4-fucosylated HMOs in human breast milk. However, this means that the assessment can only be done during lactation and if milk sample is provided by the mother.

Accordingly, it would be desirable to provide a method for predicting the levels of fucosylated HMOs which will be and/or are expressed by a human subject when lactating. Additionally or alternatively, it would be desirable to provide a method for predicting the levels of fucosylated HMOs which will be and/or are expressed by subject when lactating and which doesn't require the provision of the human breast milk expressed by that subject.

Accordingly, it would also be desirable to provide a method for predicting the levels of alpha-1,2-fucosylated HMOs which will be and/or are expressed by a human subject when lactating. Additionally or alternatively, it would be desirable to provide a method for predicting the levels of alpha-1,2-fucosylated HMOs which will be and/or are expressed by subject when lactating and which doesn't require the provision of the human breast milk expressed by that subject.

Accordingly, it would also be desirable to provide a method for predicting the levels of alpha-1,3/4-fucosylated HMOs which will be and/or are expressed by a human subject when lactating. Additionally or alternatively, it would be desirable to provide a method for predicting the levels of alpha-1,3/4-fucosylated HMOs which will be and/or are expressed by subject when lactating and which doesn't require the provision of the human breast milk expressed by that subject.

It is the object of the present invention to provide a solution to one or more of the above-mentioned problems.

### SUMMARY OF THE INVENTION

The present inventors have surprisingly found that a solution to such problem is provided by the method as described in the attached claims.

The present inventors developed a genetic score to assess the impact of genetic polymorphisms of FUT2 and FUT3 genes on the concentrations of alpha-1,2-fucosylated human milk oligosaccharides (2′FL) expressed in the milk. The inventors where able to show that the Secretor population could be divided into two sub-populations where moderate and high levels of 2′FL are expressed respectively, and that such levels can be justly predicted for a future mother's milk based on her polygenic score (prediction score with Adjusted-R²=0.58, p<6.6.10⁻⁹). A zero or negative score predicts a moderate or lower 2′FL level, while a positive score predicts a higher amount of 2′FL in the subject's milk (see Table 1 and FIG. 1). Though the Secretor status (determining 2′FL levels) is mainly dictated by single nucleotide polymorphism (SNP) rs601338, the use of our polygenic score predicts more accurately the secreted 2′FL levels than this SNP alone (p<0.001).

Accordingly, in one aspect the present invention provides a polygenic score (ps) for predicting levels of alpha-1,2-fucosylated human milk oligosaccharides secreted in breast milk by a human subject during lactation which is calculated as follows:

ps=[f(rs601338)]×−0.56304+[f(rs28362459)]×0.39504+[f(rs778986)×0.18314+[f(rs1800022)]×−0.36211+[f(rs281377)]×0.10313

wherein f represents a factor [f (SNP)] attributed to each listed SNP (single nucleotide polymorphism), the factor (fMM) being equal to 0 for all SNPs having two major alleles (MM), the factor (fMm) being equal to 1 for all SNPs having one major and one minor allele (Mm) and the factor (fmm) being equal 2 for all SNP having two minor alleles (mm).

In another aspect the present invention provides a method for attributing a polygenic score (ps) to a human subject, the method comprising:

a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);

b) Determining that the human subject belongs to the group of Non-secretors if SNP rs601338 has two minor alleles (mm) or if SNP rs601338 has two major alleles (MM) or one major and one minor allele (Mm) determining that the subject belongs to the group of Secretors and continuing with step c);

c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;
d) calculating the polygenic score (ps) for the subject by using the following equation:

ps=[f(rs601338)]×−0.56304+[f(rs28362459)]×0.39504+[f(rs778986)×0.18314+[f(rs1800022)]×−0.36211+[f(rs281377)]×0.10313.

In a further aspect, the present invention also provides a method for predicting levels of alpha-1,2-fucosylated human milk oligosaccharides secreted in breast milk by a human subject during lactation based on a polygenic score, the method comprising:

a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);

b) Determining that the human subject belongs to the group of Non-secretors if SNP rs601338 has two minor alleles (mm) or if SNP rs601338 has two major alleles (MM) or one major and one minor allele (Mm) determining that the subject belongs to the group of Secretors and continuing with step c);

c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

d) calculating the polygenic score (ps) for the subject by using the following equation:

ps=[f(rs601338)]×−0.56304+[f(rs28362459)]×0.39504+[f(rs778986)×0.18314+[f(rs1800022)]×−0.36211+[f(rs281377)]×0.10313;

e) if the polygenic score resulting from step d) is negative or zero, determining that the human subject belongs to the group of moderate secretors, while if the polygenic score resulting from step d) is positive, determining that the human subject belongs to the group of high secretors.

In another aspect the present invention also provides for a method for providing a nutritional recommendation for supplementation of alpha-1,2-fucosylated human milk oligosaccharides in the diet of an infant and/or a young child which is breast fed by a human subject, the method comprising:

a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);

b) Determining that the human subject belongs to the group of Non-secretors if SNP rs601338 has two minor alleles (mm) or if SNP has rs601338 two major alleles (MM) or one major and one minor allele (Mm) determining that the subject belongs to the group of Secretors and continuing with step c);

c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;
d) calculating the polygenic score (ps) for the subject by using the following equation:

ps=[f(rs601338)]×−0.56304+[f(rs28362459)]×0.39504+[f(rs778986)×0.18314+[f(rs1800022)]×−0.36211+[f(rs281377)]×0.10313;

e) if the polygenic score resulting from step d) is negative or zero, determining that the human subject belongs to the group of moderate secretors, while if the polygenic score resulting from step d) is positive, determining that the human subject belongs to the group of high secretors;

f) depending which group the human subject belongs to providing the following nutritional recommendation:
f′) if the human subject belongs to the group of Non-secretors, recommending that the infant consumes synthetic nutritional compositions providing a total daily intake of 2′FL ranging from an amount of 1.2 g to 3 g; or
f″) if the human subject belongs to the group of moderate secretors, recommending that the infant consumes synthetic nutritional compositions providing a total daily intake of 2′FL ranging from an amount of 0.8 g to 1.5 g; or
f′″) if the human subject belongs to the group of high secretors and the infant is breast fed only partially, recommending that the infant consumes synthetic nutritional compositions providing a total daily intake of 2′FL ranging from an amount of 0.8 g to 1.5 g.

In another aspect, the present invention provides for a data processing device comprising means for carrying out a computer implemented method to attribute a polygenic score (ps) according to the invention to a human subject.

In another aspect, the present invention provides for a data processing device comprising means for carrying out a computer implemented method to predict levels of alpha-1,2-fucosylated human milk oligosaccharides secreted in breast milk by a human subject during lactation based on a polygenic score according to the invention.

In another aspect, the present invention provides for a data processing device comprising means for carrying out a computer implemented method to provide a nutritional recommendation according to the present invention for supplementation of alpha-1,2-fucosylated human milk oligosaccharides in the diet of an infant which is breast fed by a human subject.

In a still further aspect, the present invention provides for a computer readable medium comprising instructions which, when executed by a computer, cause the computer to carry out the steps of a method to attribute a polygenic score (ps) according to the invention to a human subject.

In a still further aspect, the present invention provides for a computer readable medium comprising instructions which, when executed by a computer, cause the computer to carry out the steps of a method to predict levels of alpha-1,2-fucosylated human milk oligosaccharides secreted in breast milk by a human subject during lactation based on a polygenic score according to the invention.

In a still further aspect, the present invention provides for a computer readable medium comprising instructions which, when executed by a computer, cause the computer to carry out the steps of a method to provide a nutritional recommendation according to the present invention for supplementation of alpha-1,2-fucosylated human milk oligosaccharides in the diet of an infant which is breast fed by a human subject. In another aspect, the present invention provides for a nutritional service for providing nutritional recommendation according to the present invention for supplementation of alpha-1,2-fucosylated human milk oligosaccharides in the diet of an infant and/or a young child.

The present inventors also developed a genetic score to assess the impact of genetic polymorphisms of FUT2 and FUT3 genes on the concentrations of alpha-1,3/4-fucosylated human milk oligosaccharides (3FL) expressed in the milk. The inventors where able to show that the expressor population could be divided into two sub-populations where moderate and high levels of 3FL are expressed respectively, and that such levels can be justly predicted for a future mother's milk based on her polygenic score (prediction score with Adjusted-R²=0.78, p<6.7.10⁻¹⁴). A zero or negative score predicts a moderate or lower 3FL level, while a positive score predicts a higher amount of 3FL in the subject's milk (see Table 1 and FIG. 1). Though the expression status (determining if 3FL levels are detectable) is mainly dictated by single nucleotide polymorphisms (SNP) rs3745635, rs28362459, rs3894326, rs812936, the use of our polygenic score predicts more accurately the secreted 3FL levels than these two SNPs (p<0.001).

Accordingly, in another aspect the present invention provides a polygenic score (ps) for predicting levels of alpha-1,3/4-fucosylated human milk oligosaccharides secreted in breast milk by a human subject during lactation which is calculated as follows: ps=[f(rs516316)]×0.54808+[f(rs812936)]×−0.43406+[f(rs3894326)×−0.59029]+[f(rs1800022)]×0.66273 wherein f represents a factor [f(SNP)] attributed to each listed SNP (single nucleotide polymorphism), the factor (fMM) being equal to 0 for all SNPs having two major alleles (MM), the factor (fMm) being equal to 1 for all SNPs having one major and one minor allele (Mm) and the factor (fmm) being equal 2 for all SNP having two minor alleles (mm).

In another aspect the present invention provides a method for attributing a polygenic score (ps) to a human subject, the method comprising:

A′) Genotyping a human subject for the following SNPs: rs516316, rs812936, rs3894326, and rs1800022 and determining whether the human subject carries for each of such SNPs two major alleles (MM), one major and one minor allele (Mm) or two minor alleles (mm);

B′) Determining that the human subject belongs to the group of non-expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

D′) calculating the polygenic score (ps) for the subject by using the following equation:

ps=[f(rs516316)]×0.54808+[f(rs812936)]×−0.43406+[f(rs3894326)×−0.59029]+[f(rs1800022)]×0.66273.

In a further aspect, the present invention also provides a method for predicting levels of alpha-1,3/4-fucosylated human milk oligosaccharides secreted in breast milk by a human subject during lactation based on a polygenic score, the method comprising:
A′) Genotyping a human subject for the following SNPs: rs516316, rs812936, rs3894326, and rs1800022 and determining whether the human subject carries for each of such SNPs two major alleles (MM), one major and one minor allele (Mm) or two minor alleles (mm);

B′) Determining that the human subject belongs to the group of non expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

D′) calculating the polygenic score (ps) for the subject by using the following equation:

ps=[f(rs516316)]×0.54808+[f(rs812936)]×−0.43406+[f(rs3894326)×−0.59029]+[f(rs1800022)]×0.66273

E′) if the polygenic score resulting from step d) is negative or zero, determining that the human subject belongs to the group of moderate expressors, while if the polygenic score resulting from step d) is positive, determining that the human subject belongs to the group of high expressors.

In another aspect the present invention also provides for a method for providing a nutritional recommendation for supplementation of alpha-1,3/4-fucosylated human milk oligosaccharides in the diet of an infant and/or a young child which is breast fed by a human subject, the method comprising:

A′) Genotyping a human subject for the following SNPs: rs516316, rs812936, rs3894326, rs1800022, rs602662, rs141630650, and rs28362465 and determining whether the human subject carries for each of such SNPs two major alleles (MM), one major and one minor allele (Mm) or two minor alleles (mm);

B′) Determining that the human subject belongs to the group of non expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

d) calculating the polygenic score (ps) for the subject by using the following equation:

ps[f(rs516316)]×0.54808+[f(rs812936)]×−0.43406+[f(rs3894326)×−0.59029]+[f(rs1800022)]×0.66273;

E′) if the polygenic score resulting from step d) is negative or zero, determining that the human subject belongs to the group of moderate expressors, while if the polygenic score resulting from step d) is positive, determining that the human subject belongs to the group of high expressors;

F′) depending which group the human subject belongs to providing the following nutritional recommendation:

J′) if the human subject belongs to the group of non-expressors, recommending that the infant consumes synthetic nutritional compositions providing a total daily intake of 3FL ranging from an amount of 1 g to 3 g; or

J″) if the human subject belongs to the group of moderate expressors, recommending that the infant consumes synthetic nutritional compositions providing a total daily intake of 3FL ranging from an amount of 0.5 g to 2 g; or

J″′) if the human subject belongs to the group of high expressors and the infant is breast fed only partially, recommending that the infant consumes synthetic nutritional compositions providing a total daily intake of 3FL ranging from an amount of 0.5 g to 2 g.

In another aspect, the present invention provides for a data processing device comprising means for carrying out a computer implemented method to attribute a polygenic score (ps) according to the invention to a human subject.

In another aspect, the present invention provides for a data processing device comprising means for carrying out a computer implemented method to predict levels of alpha-1,3/4-fucosylated human milk oligosaccharides secreted in breast milk by a human subject during lactation based on a polygenic score according to the invention.

In another aspect, the present invention provides for a data processing device comprising means for carrying out a computer implemented method to provide a nutritional recommendation according to the present invention for supplementation of alpha-1,3/4-fucosylated human milk oligosaccharides in the diet of an infant which is breast fed by a human subject.

In a still further aspect, the present invention provides for a computer readable medium comprising instructions which, when executed by a computer, cause the computer to carry out the steps of a method to attribute a polygenic score (ps) according to the invention to a human subject.

In a still further aspect, the present invention provides for a computer readable medium comprising instructions which, when executed by a computer, cause the computer to carry out the steps of a method to predict levels of alpha-1,3/4-fucosylated human milk oligosaccharides secreted in breast milk by a human subject during lactation based on a polygenic score according to the invention.

In a still further aspect, the present invention provides for a computer readable medium comprising instructions which, when executed by a computer, cause the 20 computer to carry out the steps of a method to provide a nutritional recommendation according to the present invention for supplementation of alpha-1,3/4-fucosylated human milk oligosaccharides in the diet of an infant which is breast fed by a human subject.

In another aspect, the present invention provides for a nutritional service for providing nutritional recommendation according to the present invention for supplementation of alpha-1,3/4-fucosylated human milk oligosaccharides in the diet of an infant and/or a young child.

### BRIEF DESCRIPTION OF THE DRAWINGS

FIG. 1 shows 2′FL concentrations mg/L in milk of secretor mothers by polygenic score categorization in high Secretors (positive score) and moderate Secretors (zero or negative score).

FIG. 2 shows the correlation between predicted (according to the polygenic score) and observed concentrations of 2′FL mg/L secreted in the milk of the mothers.

FIG. 3 shows 3FL concentrations mg/L in milk of expressor mothers by polygenic score categorization in high expressors (positive score) and moderate expressors (zero or negative score).

FIG. 4 shows the correlation between predicted (according to the polygenic score) and observed concentrations of 3FL mg/L secreted in the milk of the mothers.

### DETAILED DESCRIPTION OF THE INVENTION

### Definitions

Within the context of the present invention, the term “human subject” or “subject” indicates a living human being, for example a woman. In one embodiment, the subject may be lactating or not lactating. In another embodiment, the subject is a lactating woman.

Within the context of the present invention the term “genotyping” means collecting a DNA sample from an individual and analysing it by laboratory methods known to the skilled person, such as sequencing or Real Time Polymerase Chain Reaction (RT-PCR), to identify the DNA sequences of a subject, in particular the genotypes on specific polymorphic positions in the genome.

Within the context of the present invention the term “single nucleotide polymorphism”, or “SNP” indicates a polymorphic position within the genome at a single nucleotide position that comprises a change of such single nucleotide which could include an adenine (A), a cytosine (C), a guanine (G) or a thymine (T) in its backbone. In most cases SNPs can take 2 different forms but there are also cases when the nucleotide can take more than 2 forms.

Within the context of the present invention, the term “allele” means the alternative form and/or forms that the nucleotide of a SNP may take.

Within the context of the present invention the term “major allele” (also indicated as “M”) means the allele that is the ancestral allele in the genome and normally the most frequent in the population.

Within the context of the present invention the term “minor allele” (also indicated as “m”) means the allele that has the alternative or mutated form(s).

Within the context of the present invention the term “polygenic score” or “ps” indicates a mathematical score calculated taking into consideration the genetic information relating to more than one SNPs.

In one embodiment of the present invention, the “polygenic score” is calculated to predict predicting levels of alpha-1,2-fucosylated human milk oligosaccharides, for example 2′-FL, secreted in breast milk by a human subject during lactation.

In another embodiment of the present invention, the “polygenic score” is calculated to predict predicting levels of alpha-1,3/4-fucosylated human milk oligosaccharides, for example 3-FL, expressed in breast milk by a human subject during lactation.

Within the context of the present invention, the term “f” or “[f (SNP)]” means a factor which is attributed to each listed SNP (single nucleotide polymorphism) for the calculation of the polygenic score. In the polygenic score, the factor (fMM) is equal to 0 for all SNPs having two major alleles (MM), the factor (fMm) is equal to 1 for all SNPs having one major and one minor allele (Mm) and the factor (fmm) is equal to 2 for all SNP having two minor alleles (mm).

Within the context of the present invention the term “alpha-1,2-fucosylated human milk oligosaccharides” indicates oligosaccharides that include an alpha-1,2-fucose group at its reducing end. Non limiting examples of such oligosaccharides are: 2′-O-Fucosyllactose (2′-FL), Lactodifucotetraose (LDFT), A-tetrasaccharide (PI), Lacto-N-fucopentaose I (LNFP-I), Lacto-N-di-fucohexaose I (LNdiFH I) and Difucosyl-lacto-N-hexaose a (DFLNHa).

Within the context of the present invention the term “alpha-1,3/4-fucosylated human milk oligosaccharides” indicates oligosaccharides that include an alpha-1,3/4-fucose group at its reducing end. Non limiting examples of such oligosaccharides are: 3-O-Fucosyllactose (3FL or 3-FL), Lactodifucotetraose (LDFT), Lacto-N-fucopentaose II (LNFP-II), Lacto-N-di-fucohexaose II (LNDFH-II) and Lacto-N-fucopentaose V (LNFP-V).

Within the context of the present invention, the term “secretory group” identifies a subject who is genetically capable of expressing substantial (for example detectable and quantifiable and/or >50 mg/L) levels of alpha-1,2-fucosylated human milk oligosaccharides during lactation.

Within the context of the present invention the term “high secretor group” is referred to a subject belonging to a group that secretes 2′FL in an amount equal or higher than 2000 mg/L in human breast milk expressed during lactation.

Within the context of the present invention the term “moderate secretor group” is referred to a subject belonging to a group that secretes 2′FL in an amount up to 2000 mg/L (or between 50 and 2000 mg/L) in human breast milk expressed during lactation.

Within the context of the present invention the term “non-secretory” or “non secretor” identifies a subject who has no substantial secretion (for example blow detection limit) of any alpha-1,2-fucosylated human milk oligosaccharides such as for example 2′-O-Fucosyllactose (2′FL), Lactodifucotetraose (LDFT), -tetrasaccharide (PI), Lacto-N-fucopentaose I (LNFP-I), Lacto-N-di-fucohexaose I (LNdiFH I), Difucosyl-lacto-N-hexaose a (DFLNHa).

In one embodiment, the term “non-secretory” or “non secretor” identifies a subject who has no substantial secretion (for example below detection limit and/or below 50 mg/L) of 2′-O-Fucosyllactose (2′FL).

Within the context of the present invention, the term “expression group” identifies a subject who is genetically capable of expressing substantial (for example detectable and quantifiable) levels of alpha-1,3/4-fucosylated human milk oligosaccharides during lactation.

Within the context of the present invention the term “high expression group” is referred to a subject belonging to a group that secretes 3FL in an amount equal or higher than 890 mg/L in human breast milk expressed during lactation.

Within the context of the present invention the term “moderate expressor group” is referred to a subject belonging to a group that secretes 3FL in an amount up to 890 mg/L (or between 450 and 890 mg/L) in human breast milk 20 expressed during lactation.

Within the context of the present invention the term “non-expression” or “non expressor” identifies a subject who has low or no substantial expression of (for example below detection limit) any alpha-1,3/4-fucosylated human milk oligosaccharides such as for example 3-O-Fucosyllactose (3FL), Lactodifucotetraose (LDFT), Lacto-N-fucopentaose II (LNFP-II), Lacto-N-di-fucohexaose II (LNDFH-II) and Lacto-N-fucopentaose V (LNFP-V).

In one embodiment, the term “non-expression” or “non expressor” identifies a subject who has low expression (for example below below 450 mg/L) or no substantial expression of (for example below detection limit) of 3-O-Fucosyllactose (3FL).

The term “infant” in the context of the present invention identifies a child under the age of 12 months, such as under the age of 9 months, particularly under the age of 6 months.

In the context of the present invention the infant may be any term infant or preterm infant. In an embodiment of the invention, the infant is selected from the group of preterm infants and term infants.

The term “term infant” refers to infants born at term or at a gestational age of 37 weeks or more.

The term “preterm infant” refers to infants who are born at a gestational age of less than 37 weeks.

In the context of the present invention, the term “birth weight” means the first weight of a newborn obtained right after birth.

Within the context of the present invention, the term “low birth weight” means a birth weight of less than 2500 g (up to and including 2499 g).

Within the context of the present invention, the term “very low birth weight” means a birth weight of less than 1500 g (up to and including 1499 g).

Within the context of the present invention, the term “extremely low birth weight” means a birth weight of less than 1000 g (up to and including 999 g).

The term “small for gestational age infant” refers to infants having a birth weight that is more than 2 standard deviations below the mean reference to a birth weight for gestational growth chart or having a birth weight that is less than the 10^{th} percentile of population-based weight data obtained from infants at the same gestational age. The term “small for gestational age infants” includes infants who are small at birth either from a constitutive or genetic origin or, as a consequence of intrauterine growth restriction.

Within the context of the present invention, the term “young children” or “toddler” indicates a child between the age of 1 and 4 years.

Within the context of the present invention, the term “nutritional composition” or “synthetic nutritional composition” means a composition which may be consumed by an infant and providing such infant with macro (for example lipids, proteins, carbohydrates) and/or micro (for example vitamins, minerals) nutrients. The synthetic nutritional composition according to the present invention is not human breast milk. Non limiting examples of nutritional compositions according to the present invention include: an infant formula, a fortified milk, a supplement and/or a milk fortifier.

The term “infant formula” as used herein refers to a nutritional composition intended for infants and as defined in Codex Alimentarius, (Codex STAN 72-1981) and Infant Specialities (incl. Food for Special Medical Purpose) as defined in Codex Alimentarius, (Codex STAN 72-1981). It also refers to a foodstuff intended for particular nutritional use by infants during the first months of life and satisfying by itself the nutritional requirements of this category of person (Article 2(c) of the European Commission Directive 91/321/EEC 2006/141/EC of 22 Dec. 2006 on infant formulae and follow-on formulae). The infant formulas encompass the starter infant formulas and the follow-up or follow-on formulas. Generally, a starter formula is for infants from birth as breast-milk substitute, and a follow-up or follow-on formula from the 6th month onwards.

The “growing-up milks” (or GUMs) are given from one year onwards. It is generally a milk-based beverage adapted and fortified for the specific nutritional needs of young children. They are nutritional compositions used for feeding children from 12 months to 2-4 years old in combination with other foods.

Within the context of the present invention, the term “fortifier” refers to a composition which comprises one or more nutrients having a nutritional benefit for infants or young children.

By the term “milk fortifier”, it is meant any composition used to fortify or supplement either human breast milk, infant formula, growing-up milk or human breast milk fortified with other nutrients. Accordingly, the human milk fortifier of the present invention can be administered after dissolution in human breast milk, infant formula, growing-up milk or human breast milk fortified with other nutrients or otherwise it can be administered as a stand-alone composition.

When administered as a stand-alone composition, the human milk fortifier can be also identified as being a “supplement”. In one embodiment, the milk fortifier of the present invention is a supplement.

By the term “human milk fortifier”, it is meant any composition used to fortify or supplement human breast milk, or human breast milk fortified with other nutrients. The “human milk fortifier” according to the present invention may be intended to be administered to infants who were born term, with normal weight or small for gestational age, or born preterm, with very low birth weight (VLBW) or with extremely low birth weight (ELBW).

The milk fortifier according to the present invention may be in powder of liquid form.

Milk fortifier compositions having a liquid form presents some particular benefits. For example, liquid formulations might be more convenient if coupled with a packaging that delivers calibrated drops of a certain weight or volume. In addition, liquid formulations are easier to mix with the compositions to be fortified, whereas the powder ones can, in some cases, form lumps.

### Embodiments of the Invention

### Polygenic Score (alpha-1,2-fucosylated Human Milk Oligosaccharides)

Accordingly, in one aspect the present invention provides a polygenic score (ps) for predicting levels of alpha-1,2-fucosylated human milk oligosaccharides secreted in breast milk by a human subject during lactation which is calculated as follows:

ps=[f(rs601338)]×−0.56304+[f(rs28362459)]×0.39504+[f(rs778986)×0.18314+[f(rs1800022)]×−0.36211+[f(rs281377)]×0.10313

wherein f represents a factor [f (SNP)] attributed to each listed SNP (single nucleotide polymorphism), the factor (fMM) being equal to 0 for all SNPs having two major alleles (MM), the factor (fMm) being equal to 1 for all SNPs having one major and one minor allele (Mm) and the factor (fmm) being equal 2 for all SNP having two minor alleles (mm).

According to the present invention, the polygenic factor is calculated by attributing a factor [f (SNP)] to each listed SNP on the basis of presence or absence of a specific mutated gene in the subject.

More specifically, SNP rs601338 on FUT2 may take either a guanine (major allele) or an adenine (minor allele) resulting to a change from phenylalanine to a stop codon and an early termination of the protein chain. SNP rs28362459 on FUT3 may take either an adenine (major) or a cytosine, guanine, thymine (minor) resulting to a change of leucine to arginine in the protein change. SNP rs778986 on FUT3 may take either an adenine (major) or a guanine (minor) resulting to a change of threonine to methionine in the protein change. SNP rs1800022 on FUT2 may take a cytosine (major) or a thymine (minor) resulting to a change of arginine to cysteine in the protein change. SNP rs281377 on FUT2 may take either a cytosine (major) or a thymine (minor) resulting to no change of amino acid which is in both cases asparagine.

Table 2 reported below summarizes the possible mutations of the SNPs and their impact on the expressed protein.

TABLE 2

| | | Chromosome | | | Amino | Position |
|---|---|---|---|---|---|---|
| SNP | Gene | position | Function | Allele | acid change | in protein |
| rs28362459 | FUT3 | 5844781 | Missense | A > C/G/T | L > R | 20 |
| rs1800022 | FUT2 | 48703368 | Missense | C > T | R > C | 138 |
| rs601338 | FUT2 | 48703417 | Stop gain | G > A | W > *Ter | 154 |
| | | | variant | | | |
| rs778986 | FUT3 | 5844526 | Missense | A > G | T > M | 105 |
| rs281377 | FUT2 | 48703346 | Synonymous | C > T | N > N | 130 |

Thus in one embodiment, [f (rs601338)] is zero when rs601338 has two guanine, [f (rs601338)] is 1 when rs601338 has one guanine and one adenine or [f (rs601338)] is 2 when rs601338 has two adenine.

In one embodiment, [f (rs28362459)] is zero when rs601338 has two adenine, [f (rs28362459)] is 1 when rs28362459 has one adenine and one cytosine, guanine or thymine or [f (rs28362459)] is 2 when rs28362459 has two cytosine, guanine or thymine.

In one embodiment, [f (rs778986)] is zero when rs778986 has two adenine, [f (rs778986)] is 1 when rs778986 has one adenine and one guanine or [f (rs778986)] is 2 when rs778986 has two guanine.

In one embodiment, [f (rs1800022)] is zero when rs1800022 has two cytosine, [f (rs1800022)] is 1 when rs1800022 has one cytosine and one thymine or [f (rs1800022)] is 2 when rs1800022 has two thymine.

In one embodiment, [f (rs281377)] is zero when rs281377 has two cytosine, [f (rs281377)] is 1 when rs281377 has one cytosine and one thymine or [f (rs281377)] is 2 when rs281377 has two thymine.

### Polygenic Score (alpha-1,3/4-fucosylated Human Milk Oligosaccharides)

Accordingly, in one aspect the present invention provides a polygenic score (ps) for predicting levels of alpha-1,3/4-fucosylated human milk oligosaccharides secreted in breast milk by a human subject during lactation which is calculated as follows:

ps==[f(rs516316)]×0.54808+[f(rs812936)]×−0.43406+[f(rs3894326)×−0.59029]+[f(rs1800022)]×0.66273

wherein f represents a factor [f (SNP)] attributed to each listed SNP (single nucleotide polymorphism), the factor (fMM) being equal to 0 for all SNPs having two major alleles (MM), the factor (fMm) being equal to 1 for all SNPs having one major and one minor allele (Mm) and the factor (fmm) being equal 2 for all SNP having two minor alleles (mm).

According to the present invention, the polygenic factor is calculated by attributing a factor [f (SNP)] to each listed SNP on the basis of presence or absence of a specific mutated gene in the subject.

More specifically, SNP rs516316 on FUT2 may take either a guanine (major allele) or an cytosine (minor allele) resulting to no of the protein chain (intronic SNP). SNP rs812936 on FUT3 may take either an guanine (major) or a adenine, cytosine (minor) resulting to a change of tryptophan to arginine or glycine in the protein change. SNP rs3894326 on FUT3 may take either an adenine (major) or a cytosine, thymine (minor) resulting to a change of isoleucine to arginine, lysine in the protein change. SNP rs1800022 on FUT3 may take a cytosine (major) or a thymine (minor) resulting to a change of arginine to cysteine in the protein change.

Table 3 reported below summarizes the possible mutations of the SNPs and their impact on the expressed protein.

TABLE 3

| | | Chromosome | | | Amino | Position |
|---|---|---|---|---|---|---|
| SNP | Gene | position | Function | Allele | acid change | in protein |
| rs516316 | FUT2 | 48702888 | Intron | G > C | NA | NA |
| | | | Variant | | | |
| rs812936 | FUT3 | 5844638 | Missense | G > AC | W > R/G | 68 |
| rs3894326 | FUT3 | 5843773 | Missense | A > C/T | I > K | 356 |
| rs1800022 | FUT2 | 48703368 | Missense | C > T | R > C | 138 |

Thus in one embodiment, [f (rs516316)] is zero when rs516316 has two guanine, [f (rs516316)] is 1 when rs516316 has one guanine and one cytosine, [f (rs516316)] is 2 when rs516316 has two cytosines.

In one embodiment, [f (rs812936)] is zero when rs812936 has two guanine, [f (rs812936)] is 1 when rs28362459 has one guanine and one adenine, or cytosine, [f (rs28362459)] is 2 when rs28362459 has two adenine or cytosine.

In one embodiment, [f (rs3894326)] is zero when rs3894326 has two adenine, [f (rs3894326] is 1 when rs3894326 has one adenine and one cytosine or thymine, [f (rs3894326)] is 2 when rs3894326 has two cytosine or thymine.

In one embodiment, [f (rs1800022)] is zero when rs1800022 has two cytosine, [f (rs1800022)] is 1 when rs1800022 has one cytosine and one thymine, [f (rs1800022)] is 2 when rs1800022 has two thymine.

### Method for Attributing a Polygenic Score to a Subject (alpha-1,2-fucosylated Human Milk Oligosaccharides)

In another aspect the present invention provides a method for attributing a polygenic score (ps) to a human subject, the method comprising:

e) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);

f) Determining that the human subject belongs to the group of Non-secretors if SNP rs601338 has two minor alleles (mm) or if SNP rs601338 has two major alleles (MM) or one major and one minor allele (Mm) determining that the subject belongs to the group of Secretors and continuing with step c);

g) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

h) calculating the polygenic score (ps) for the subject by using the following equation:

ps=[f(rs601338)]×−0.56304+[f(rs28362459)]×0.39504+[f(rs778986)×0.18314+[f(rs1800022)]×−0.36211+[f(rs281377)]×0.10313.

In one embodiment, the polygenic score is attributed predicting levels of alpha-1,2-fucosylated human milk oligosaccharides in a subject.

In one embodiment of the present invention, the subject is a woman for example a woman intending to become pregnant, woman who is pregnant and/or a lactating woman.

In another aspect, the present invention provides for a data processing device comprising means for carrying out a computer implemented method according to the invention to attribute a polygenic score (ps) to a human subject.

In one embodiment of the present invention, the data processing device may be a computer or a smartphone.

In a still further aspect, the present invention provides for a computer readable medium comprising instructions which, when executed by a computer, cause the computer to carry out the steps of a method to attribute a polygenic score (ps) according to the invention to a human subject.

In one embodiment of the present invention, the method for attributing a polygenic score (ps) to a human subject for alpha-1,2-fucosylated human milk oligosaccharides is performed in combination with a method for attributing a polygenic score (ps) to a human subject for alpha-1,3/4-fucosylated human milk oligosaccharides.

Within the context of the present invention, the term “performed in combination with” means that the two methods may be run in parallel, sequentially or separately. Preferably, the methods are run in parallel or sequentially.

### Method for Attributing a Polygenic Score to a Subject (alpha-1,3/4-fucosylated Human Milk Oligosaccharides)

In another aspect the present invention provides a method for attributing a polygenic score (ps) to a human subject, the method comprising:

A′) Genotyping a human subject for the following SNPs: rs516316, rs812936, rs3894326, and rs1800022 and determining whether the human subject carries for each of such SNPs two major alleles (MM), one major and one minor allele (Mm) or two minor alleles (mm);
B′) Determining that the human subject belongs to the group of non expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);
C′) attributing a factor [f (SNP)] to all SNPs, the factor (fMM) being equal 5 to 0 for all SNPs resulting to be MM, the factor (fMm) being equal to 1 for all SNPs resulting to be Mm and the factor (fmm) being equal 2 for all genes resulting to be mm from step a) for the human subject;
D′) calculating the polygenic score (ps) for the subject by using the following equation:

ps=[f(rs516316)]×0.54808+[f(rs812936)]×−0.43406+[f(rs3894326)×−0.59029]+[f(rs1800022)]×0.66273

In one embodiment, the polygenic score is attributed predicting levels of alpha-1,3/4-fucosylated human milk oligosaccharides in a subject.

In one embodiment of the present invention, the subject is a woman for example a woman intending to become pregnant, woman who is pregnant and/or a lactating woman.

In another aspect, the present invention provides for a data processing device comprising means for carrying out a computer implemented method according to the invention to attribute a polygenic score (ps) to a human subject.

In one embodiment of the present invention, the data processing device may be a computer or a smartphone.

In a still further aspect, the present invention provides for a computer readable medium comprising instructions which, when executed by a computer, cause the computer to carry out the steps of a method to attribute a polygenic score (ps) according to the invention to a human subject.

### Method for Predicting Levels of alpha-1,2-fucosylated Human Milk Oligosaccharides Secreted in Breast Milk by a Human Subject during Lactation

In a further aspect, the present invention also provides a method for predicting levels of alpha-1,2-fucosylated human milk oligosaccharides secreted in breast milk by a human subject during lactation based on a polygenic score, the method comprising:

a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);

b) Determining that the human subject belongs to the group of Non-secretors if SNP rs601338 has two minor alleles (mm) or if SNP rs601338 has two major alleles (MM) or one major and one minor allele (Mm) determining that the subject belongs to the group of Secretors and continuing with step c);

c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

d) calculating the polygenic score (ps) for the subject by using the following equation:

ps=[f(rs601338)]×−0.56304+[f(rs28362459)]×0.39504+[f(rs778986)×0.18314+[f(rs1800022)]×−0.36211+[f(rs281377)]×0.10313;

e) if the polygenic score resulting from step d) is negative or zero, determining that the human subject belongs to the group of moderate secretors, while if the polygenic score resulting from step d) is positive, determining that the human subject belongs to the group of high Secretors.

In one embodiment of the present invention, the subject is a woman for example a pregnant and/or a lactating woman.

In one embodiment, the alpha-1,2-fucosylated human milk oligosaccharides is 2′FL.

In another aspect, the present invention provides for a data processing device comprising means for carrying out a computer implemented method to predict levels of alpha-1,2-fucosylated human milk oligosaccharides secreted in breast milk by a human subject during lactation based on a polygenic score according to the invention.

In one embodiment of the present invention, the data processing device may be a computer or a smartphone.

In a still further aspect, the present invention provides for a computer readable medium comprising instructions which, when executed by a computer, cause the computer to carry out the steps of a method to predict levels of alpha-1,2-fucosylated human milk oligosaccharides secreted in breast milk by a human subject during lactation based on a polygenic score according to the invention.

In one embodiment of the present invention, the method for predicting levels of alpha-1,2-fucosylated human milk oligosaccharides is performed in combination with a method for predicting levels of alpha-1,3/4-fucosylated human milk oligosaccharides.

Within the context of the present invention, the term “performed in combination with” means that the two methods may be run in parallel, sequentially or separately. Preferably, the methods are run in parallel or sequentially.

### Method for Predicting Levels of alpha-1,3/4-fucosylated Human Milk Oligosaccharides Secreted in Breast Milk by a Human Subject during Lactation

In a further aspect, the present invention also provides a method for predicting levels of alpha-1,3/4-fucosylated human milk oligosaccharides secreted in breast milk by a human subject during lactation based on a polygenic score, the method comprising:

A′) Genotyping a human subject for the following SNPs: rs516316, rs812936, rs3894326, and rs1800022 and determining whether the human subject carries for each of such SNPs two major alleles (MM), one major and one minor allele (Mm) or two minor alleles (mm);

B′) Determining that the human subject belongs to the group of non expressors if SNP rs601338 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

D′) calculating the polygenic score (ps) for the subject by using the following equation:

ps=[f(rs516316)]×0.54808+[f(rs812936)]×−0.43406+[f(rs3894326)×−0.59029]+[f(rs1800022)]×0.66273;

E′) if the polygenic score resulting from step d) is negative or zero, determining that the human subject belongs to the group of moderate expressors, while if the polygenic score resulting from step d) is positive, determining that the human subject belongs to the group of high expressors.

In one embodiment of the present invention, the subject is a woman for example a pregnant and/or a lactating woman.

In one embodiment, the alpha-1,3/4-fucosylated human milk oligosaccharides is 3FL.

In another aspect, the present invention provides for a data processing device comprising means for carrying out a computer implemented method to predict levels of alpha-1,3/4-fucosylated human milk oligosaccharides secreted in breast milk by a human subject during lactation based on a polygenic score according to the invention.

In one embodiment of the present invention, the data processing device may be a computer or a smartphone.

In a still further aspect, the present invention provides for a computer readable medium comprising instructions which, when executed by a computer, cause the computer to carry out the steps of a method to predict levels of alpha-1,3/4-fucosylated human milk oligosaccharides secreted in breast milk by a human subject during lactation based on a polygenic score according to the invention.

### Method to Provide a Nutritional Recommendation for alpha-1,2-fucosylated Human Milk Oligosaccharides

In another aspect the present invention also provides for a method for providing a nutritional recommendation for supplementation of alpha-1,2-fucosylated human milk oligosaccharides in the diet of an infant and/or a young child which is breast fed by a human subject, the method comprising:

a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);

b) Determining that the human subject belongs to the group of Non-secretors if SNP rs601338 has two minor alleles (mm) or if SNP rs601338 has two major alleles (MM) or one major and one minor allele (Mm) determining that the subject belongs to the group of Secretors and continuing with step c);

c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

d) calculating the polygenic score (ps) for the subject by using the following equation:

ps=[f(rs601338)]×−0.56304+[f(rs28362459)]×0.39504+[f(rs778986)×0.18314+[f(rs1800022)]×−0.36211+[f(rs281377)]×0.10313;

e) if the polygenic score resulting from step d) is negative or zero, determining that the human subject belongs to the group of moderate secretors, while if the polygenic score resulting from step d) is positive, determining that the human subject belongs to the group of high Secretors;

f) depending which group the human subject belongs to providing the following nutritional recommendation:
f′) if the human subject belongs to the group of non-secretors, recommending that the infant and/or young child consumes synthetic nutritional compositions providing a total daily intake of 2′FL ranging from an amount of 1.2 g to 3 g; or
   f″) if the human subject belongs to the group of moderate Secretors, recommending that the infant and/or young child consumes synthetic nutritional compositions providing a total daily intake of 2′FL ranging from an amount of 0.8 g to 1.5 g; or
   f′″) if the human subject belongs to the group of high secretors and the infant and/or the young child is breast fed only partially, recommending that the infant and/or the young child consumes synthetic nutritional compositions providing a total daily intake of 2′FL ranging from an amount of 0.8 g to 1.5 g.

In one embodiment of the present invention, the subject is a woman for example a pregnant and/or a lactating woman.

In one embodiment, the alpha-1,2-fucosylated human milk oligosaccharides is 2′FL.

In one embodiment, the infant and/or a young child is an infant.

In one embodiment of the present invention, consumption of 2′FL is recommended in combination with consumption of DiFL.

In another aspect, the present invention provides for a data processing device comprising means for carrying out a computer implemented method to provide a nutritional recommendation according to the present invention for supplementation of alpha-1,2-fucosylated human milk oligosaccharides in the diet of an infant which is breast fed by a human subject.

In one embodiment of the present invention, the data processing device may be a computer or a smartphone.

In a still further aspect, the present invention provides for a computer readable medium comprising instructions which, when executed by a computer, cause the computer to carry out the steps of a method to provide a nutritional recommendation according to the present invention for supplementation of alpha-1,2-fucosylated human milk oligosaccharides in the diet of an infant which is breast fed by a human subject.

In one embodiment of the present invention, the method for providing a nutritional recommendation for alpha-1,2-fucosylated human milk oligosaccharides is performed in combination with a method for providing a nutritional recommendation for alpha-1,3/4-fucosylated human milk oligosaccharides.

Within the context of the present invention, the term “performed in combination with” means that the two methods may be run in parallel, sequentially or separately. Preferably, the methods are run in parallel or sequentially.

### Method to Provide a Nutritional Recommendation for alpha-1,3/4-fucosylated Human Milk Oligosaccharides

In another aspect the present invention also provides for a method for providing a nutritional recommendation for supplementation of alpha-1,3/4-fucosylated human milk oligosaccharides in the diet of an infant and/or a young child which is breast fed by a human subject, the method comprising:

A′) Genotyping a human subject for the following SNPs: rs516316, rs812936, rs3894326, and rs1800022 and determining whether the human subject carries for each of such SNPs two major alleles (MM), one major and one minor allele (Mm) or two minor alleles (mm);

B′) Determining that the human subject belongs to the group of non expressors if SNP rs601338 has two minor alleles (mm) or if any of the SNPs rs3745635, rs28362459, rs3894326, rs812936 two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

D′) calculating the polygenic score (ps) for the subject by using the following equation:

ps=[f(rs516316)]×0.54808+[f(rs812936)]×−0.43406+[f(rs3894326)×−0.59029]+[f(rs1800022)]×0.66273;

E′) if the polygenic score resulting from step d) is negative or zero, determining that the human subject belongs to the group of moderate expressors, while if the polygenic score resulting from step d) is positive, determining that the human subject belongs to the group of high expressors;

F′) depending which group the human subject belongs to providing the following nutritional recommendation:

J′) if the human subject belongs to the group of non-expressors, recommending that the infant and/or young child consumes synthetic nutritional compositions providing a total daily intake of 3FL ranging from an amount of 1 g to 3 g; or

J″) if the human subject belongs to the group of moderate expressors, recommending that the infant and/or young child consumes synthetic nutritional compositions providing a total daily intake of 3FL ranging from an amount of 0.5 g to 2 g; or

J″′) if the human subject belongs to the group of high expressors and the infant and/or the young child is breast fed only partially, recommending that the infant and/or the young child consumes synthetic nutritional compositions providing a total daily intake of 3FL ranging from an amount of 0.5 g to 2 g.

In one embodiment of the present invention, the subject is a woman for example a pregnant and/or a lactating woman. In one embodiment, the alpha-1,3/4-fucosylated human milk oligosaccharides is 3FL. In one embodiment, the infant and/or a young child is an infant.

In another aspect, the present invention provides for a data processing device comprising means for carrying out a computer implemented method to provide a nutritional recommendation according to the present invention for supplementation of alpha-1,3/4-fucosylated human milk oligosaccharides in the diet of an infant which is breast fed by a human subject.

In one embodiment of the present invention, the data processing device may be a computer or a smartphone.

In a still further aspect, the present invention provides for a computer readable medium comprising instructions which, when executed by a computer, cause the computer to carry out the steps of a method to provide a nutritional recommendation according to the present invention for supplementation of alpha-1,3/4-fucosylated human milk oligosaccharides in the diet of an infant which is breast fed by a human subject.

### Nutritional Service for alpha-1,2-fucosylated Human Milk Oligosaccharides

In one aspect of the present invention, a nutritional service is provided for providing nutritional recommendation according to the present invention for supplementation of alpha-1,2-fucosylated human milk oligosaccharides in the diet of an infant and/or a young child.

According to one embodiment of the present invention, a nutritional service is provided whereby women intending to be pregnant, pregnant women and/or lactating mothers may provide one saliva sample to assess their Secretor group (especially during pregnancy or lactation). After sample genotyping and attribution to a secretory group according to the polygenic score of the present invention, women intending to become pregnant, pregnant women or lactating mothers will receive information on their secretion type or Secretory group (Non-secretor, low/moderate Secretor, high Secretor). For each secretory group a different alpha-1,2-fucosylated human milk oligosaccharides supplementation regime will be recommended for the infants.

For each infant feeding scenario (breastfeeding, mixed feeding or infant formula feeding) mothers will also receive a recommendation for infant alpha-1,2-fucosylated human milk oligosaccharides supplementation based on their secretory group. For each scenario they will also receive the information on HMO-related health benefits for their infant and on the importance to personalize the HMO intake by their infants.

If the assessment is made during pregnancy, once the baby is born the feeding plan can be revisited and recommendation finalized by selecting the appropriate products: IF and supplement if necessary. In case of change of the feeding plan during lactation, the nutritional recommendation may be modified based on the new scenario.

The nutritional service may implemented and provided on a computer machine, tablet or smartphone, for example in the form of a mobile application or of a web based platform.

As discussed above, knowing the levels of alpha-1,2-fucosylated human milk oligosaccharides which are and/or will be secreted by a subject when lactating is important to determine whether a supplementation for such alpha-1,2-fucosylated human milk oligosaccharides is considered necessary and if so which are the recommended levels of for such alpha-1,2-fucosylated human milk oligosaccharides to be administered.

### Nutritional Service for Supplementation of alpha-1,3/4-fucosylated Human Milk Oligosaccharides

In one aspect of the present invention, a nutritional service is provided for providing nutritional recommendation according to the present invention for supplementation of alpha-1,3/4-fucosylated human milk oligosaccharides in the diet of an infant and/or a young child.

According to one embodiment of the present invention, a nutritional service is provided whereby women intending to be pregnant, pregnant women and/or lactating mothers may provide one saliva sample to assess their expression group (especially during pregnancy or lactation). After sample genotyping and attribution to a expression group according to the polygenic score of the present invention, women intending to become pregnant, pregnant women or lactating mothers will receive information on their expression type or expression group (non expressor, low/moderate expressor, high expressor). For each expression group a different alpha-1,3/4-fucosylated human milk oligosaccharides supplementation regime will be recommended for the infants.

For each infant feeding scenario (breastfeeding, mixed feeding or infant formula feeding) mothers will also receive a recommendation for infant alpha-1,3/4-fucosylated human milk oligosaccharides supplementation based on their expression group. For each scenario they will also receive the information on HMO related health benefits for their infant and on the importance to personalize the HMO intake by their infants.

If the assessment is made during pregnancy, once the baby is born the feeding plan can be revisited and recommendation finalized by selecting the appropriate products: IF and supplement if necessary. In case of change of the feeding plan during lactation, the nutritional recommendation may be modified based on the new scenario.

The nutritional service may implemented and provided on a computer machine, tablet or smartphone, for example in the form of a mobile application or of a web based platform.

As discussed above, knowing the levels of alpha-1,3/4-fucosylated human milk oligosaccharides which are and/or will be expressed by a subject when lactating is important to determine whether a supplementation for such alpha-1,3/4-fucosylated human milk oligosaccharides is considered necessary and if so which are the recommended levels of for such alpha-1,3/4-fucosylated human milk oligosaccharides to be administered.

### Additional Embodiments

The following additional embodiments are provided by the present invention:

i). A polygenic score (ps) for predicting levels of alpha-1,3/4-fucosylated human milk oligosaccharides secreted in breast milk by a human subject during lactation which is calculated as follows:

ps=[f(rs516316)]×0.54808+[f(rs812936)]×−0.43406+[f(rs3894326)×−0.59029]+[f(rs1800022)]×0.66273

wherein f represents a factor [f (SNP)] attributed to each listed SNP (single nucleotide polymorphism), the factor (fMM) being equal to 0 for all SNPs having two major alleles (MM), the factor (fMm) being equal to 1 for all SNPs having one major and one minor allele (Mm) and the factor (fmm) being equal 2 for all SNP having two minor alleles (mm).

ii). Method for attributing a polygenic score (ps) according to embodiment i) to a human subject, the method comprising:

A′) Genotyping a human subject for the following SNPs: rs516316, rs812936, rs3894326, and rs1800022 and determining whether the human subject carries for each of such SNPs two major alleles (MM), one 20 major and one minor allele (Mm) or two minor alleles (mm);

B′) Determining that the human subject belongs to the group of non expressors if any of the SNPs rs3745635, rs28362459, rs3894326, rs812936 has two minor alleles (mm) or if SNP rs601338 has two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject

D′) calculating the polygenic score (ps) for the subject by using the following equation:

ps=[f(rs516316)]×0.54808+[f(rs812936)]×−0.43406+[f(rs3894326)×−0.59029]+[f(rs1800022)]×0.66273.

iii). Method for predicting levels of alpha-1,3/4-fucosylated human milk oligosaccharides secreted in breast milk by a human subject during lactation based on a polygenic score as described in embodiment i), the method comprising:

A′) Genotyping a human subject for the following SNPs: s516316, rs812936, rs3894326, and rs1800022 and determining whether the human subject carries for each of such SNPs two major alleles (MM), one major and one minor allele (Mm) or two minor alleles (mm);

B′) Determining that the human subject belongs to the group of non expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

D′) calculating the polygenic score (ps) for the subject by using the following equation:

ps[f(rs516316)]×0.54808+[f(rs812936)]×−0.43406+[f(rs3894326)×−0.59029]+[f(rs1800022)]×0.66273;

E′) if the polygenic score resulting from step d) is negative or zero, determining that the human subject belongs to the group of moderate expressors, while if the polygenic score resulting from step d) is positive, determining that the human subject belongs to the group of high expressors.

iv). Method for providing a nutritional recommendation for supplementation of alpha-1,3/4-fucosylated human milk oligosaccharides in the diet of an infant which is breast fed by a human subject, the method comprising:

A′) Genotyping a human subject for the following SNPs: rs516316, rs812936, rs3894326, and rs1800022 and determining whether the human subject carries for each of such SNPs two major alleles (MM), one major and one minor allele (Mm) or two minor alleles (mm);

B′) Determining that the human subject belongs to the group of non expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);

C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;

D′) calculating the polygenic score (ps) for the subject by using the following equation:

ps=[f(rs516316)]×0.54808+[f(rs812936)]×−0.43406+[f(rs3894326)×−0.59029]+[f(rs1800022)]×0.66273;

E′) if the polygenic score resulting from step d) is negative or zero, determining that the human subject belongs to the group of moderate expressors, while if the polygenic score resulting from step d) is positive, determining that the human subject belongs to the group of high expressors;

F′) depending on which group the human subject belongs to providing the following nutritional recommendation:
J′) if the human subject belongs to the group of non expressors, recommending that the infant consumes synthetic nutritional compositions providing a total daily intake of 3FL ranging from an amount of 1 g to 3 g; or
J″) if the human subject belongs to the group of moderate expressors, recommending that the infant consumes synthetic nutritional compositions providing a total daily intake of 3FL ranging from an amount of 0.5 g to 2 g; or
J″″) if the human subject belongs to the group of high expressors and the infant is breast fed only partially, recommending that the infant consumes synthetic nutritional compositions providing a total daily intake of 3FL ranging from an amount of 0.5 g to 2 g.

v). A polygenic score or method according to anyone of embodiment i) to iv) wherein the alpha-1,3/4-fucosylated human milk oligosaccharides is 3FL.

vi). Method according to anyone of embodiments ii) to v) wherein the human subject is a woman, for example a woman who is intending to get pregnant, a pregnant woman and/or a lactating woman.

vii). A data processing device comprising means for carrying out a computer implemented method according to anyone of embodiments ii) to vi).

viii). A data processing device according to embodiment vii), wherein the data processing device is a computer machine, a tablet and/or smartphone.

ix). A nutritional service for providing nutritional recommendation according to the method of anyone of embodiments iii) to vi) for supplementation of alpha-1,3/4-fucosylated human milk oligosaccharides in the diet of an infant and/or a young child.

### EXPERIMENTAL SECTION

### Determination of a Polygenic Score to Predict Levels of alpha-1,2-fucosylated Human Milk Oligosaccharides to be Secreted by Future Mothers

### Study Population

LIFE Child is a longitudinal epidemiological childhood cohort study initiated in 2011 in Leipzig, Germany. The study aims to follow children from pregnancy into young adulthood and determine risk and resilience factors for healthy development. The study is described in detail elsewhere (Quante, Hesse et al. 2012, Poulain, Baber et al. 2017). In the child's first year of life, visits are scheduled at the age of three, six, and twelve months of life. Between 2011 and 2015, 156 lactating mothers visited the study center providing saliva samples and 156 milk samples at the 3-months visit, 122 at the 6-months visit, and 28 at the 12-months visit. Mothers were aged between 23 and 42 years at the child's birth. Babies were born between the 31st and 41st week of pregnancy, with nine children born before the completed 37th week 1×31st, 2×35th, 6×37th week.

### Sequencing Samples

DNA quantification was performed using the picogreen ultra-sensitive fluorescent nucleic acid stain for quantitating double-stranded DNA Life Technologies on all DNA samples collected for saliva.

Each sample is normalized at 10ng/μL. 1 HapMap NA18523 positive sample and 1 No Template Control are added to each plate. FUT2 and FUT3 amplicons are prepared using the Kapa HiFi PCR kit following the manufacturer instructions.

Template DNA starting material is 50 ng of genomic DNA and 30 cycles with 60° C. melting temperature are performed. Tagged paired-end Nextera libraries were prepared on these amplicons and sequenced on Illumina short reads sequencing platform. The quality and quantity of each sample libraries were controlled by gel electrophoresis.

### HMO Determination

HMOs were analyzed according to the method of Austin & Benet [10]. Quantification of 2′FL and LNFP-I was performed against genuine standards purchased from Elicity Crolles, France. All other HMOs were quantified against maltotriose Sigma, Buchs, Switzerland as a surrogate standard assuming equimolar response factors.

### Polygenic Score to Predict High or Low Abundance of 2′FL in Milk of Secretor Mothers

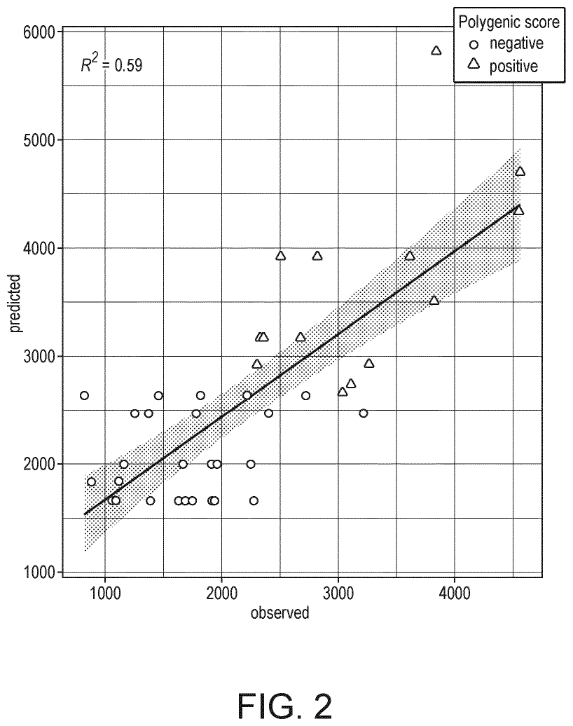

The selection of SNPs is performed by stepwise-selection of Generalized Linear Model (GLMs) in both directions. The selected model has been trained on a training dataset 200 repeats of 40-Fold CV. The evaluation of the model was performed on an independent test dataset. The selected SNPs were included in an algorithm to compute a genetic score. The genetic score was defined as the sum of the alleles for the SNPs selected in the model. Then we regressed the 2′FL concentration with the genetic score on a training set to define the prediction model. Eventually we tested the prediction on an independent test dataset. We showed the genetic score is able to predict the levels on 2′FL with an adjusted R-Square to 0.58 (see Table 1 and FIG. 2).

TABLE 1
Summary table for the SNP included in the genetic score.

| | | Standard | |
|---|---|---|---|
| SNP | Coefficient | Error | P value |
| rs601338 | −0.56304 | 0.06936 | <0.001 |
| rs28362459 | 0.39504 | 0.06534 | <0.001 |
| rs778986 | 0.18314 | 0.04536 | <0.001 |
| rs1800022 | −0.36211 | 0.14316 | 0.013 |
| rs281377 | 0.10313 | 0.05702 | 0.074 |

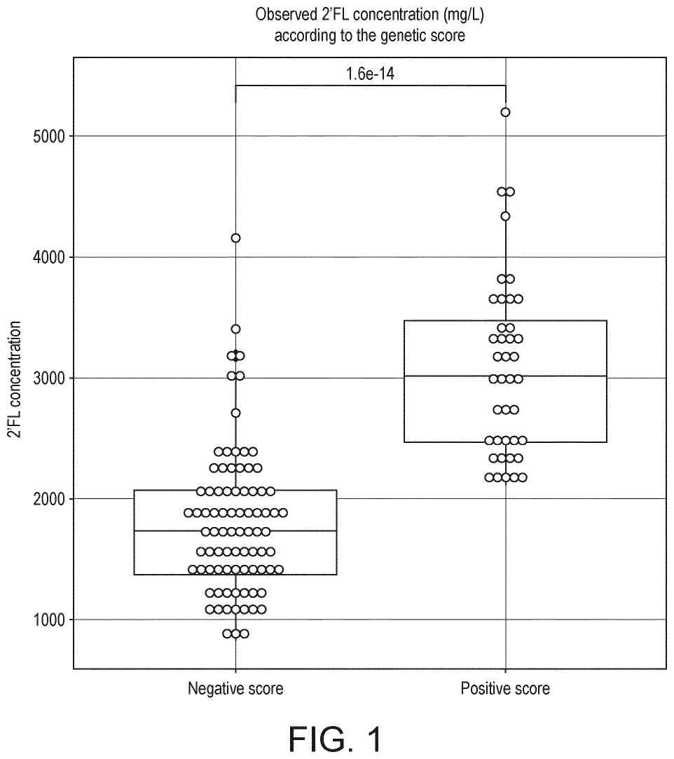

It was thus shown that Secretor population could be divided into two sub-populations with moderate and high levels of 2′FL. The polygenic model proposed could justly predict the 2′FL levels secreted in mothers. A zero or negative score predicts a moderate 2′FL level, while a positive score predicts a high amount of 2′FL (FIG. 1 and FIG. 2). Though 2′FL levels are mainly associated with rs601338 polymorphism and heterozygous mothers are predominantly represented in the moderate level group, our polygenic score significantly outperforms predictions based on rs601338 alone p<0.001.

### Example 2

### Prediction of Levels of 2-FL which Will be Secreted by a Pregnant Woman at the Time of Lactation

DNA extracted from a saliva sample of a pregnant woman was genotyped for the following SNPs: rs601338, rs28362459, rs778986, rs1800022 and rs281377. The following model is applied to obtain a polygenic score for the woman:

ps=f (rs601338)×−0.56304+f (rs28362459)×0.39504+f (rs778986)×0.18314+f (1800022)×−0.36211+f (rs281377)×0.10313, wherein factor f is attributed for each SNP as follows: the factor (fMM) is equal to 0 for all SNPs having two major alleles, the factor (fMm) is equal to 1 for all SN Ps resulting to have one major and one minor allele and the factor (fmm) being equal 2 for all genes having 2 minor alleles.

If the polygenic score obtained is positive, then the woman is predicted to secrete levels of 2′FL equal or higher than 2000 mg/L during lactation; If the polygenic score obtained is negative, then the woman is predicted to secrete levels of 2′FL lower than 2000 mg/L during lactation.

### Example 3

### Determination of a Polygenic Score to Predict Levels of alpha-1,3/4-fucosylated Human Milk Oligosaccharides to be Secreted by Future Mothers

### Study Population

LIFE Child is a longitudinal epidemiological childhood cohort study initiated in 2011 in Leipzig, Germany. The study aims to follow children from pregnancy into young adulthood and determine risk and resilience factors for healthy development. The study is described in detail elsewhere (Quante, Hesse et al. 2012, Poulain, Baber et al. 2017). In the child's first year of life, visits are scheduled at the age of three, six, and twelve months of life. Between 2011 and 2015, 156 lactating mothers visited the study center providing saliva samples and 156 milk samples at the 3-months visit, 122 at the 6-months visit, and 28 at the 12-months visit. Mothers were aged between 23 and 42 years at the child's birth. Babies were born between the 31st and 41st week of pregnancy, with nine children born before the completed 37th week 1×31st, 2×35th, 6×37th week.

### Sequencing Samples

DNA quantification was performed using the picogreen ultra-sensitive fluorescent nucleic acid stain for quantitating double-stranded DNA Life Technologies on all DNA samples collected for saliva.

Each sample is normalized at 10 ng/μL. 1 HapMap NA18523 positive sample and 1 No Template Control are added to each plate. FUT2 and FUT3 amplicons are prepared using the Kapa HiFi PCR kit following the manufacturer instructions. Template DNA starting material is 50 ng of genomic DNA and cycles with 60° C. melting temperature are performed. Tagged paired-end Nextera libraries were prepared on these amplicons and sequenced on Illumina short reads sequencing platform. The quality and quantity of each sample libraries were controlled by gel 20 electrophoresis.

### HMO Determination

HMOs were analyzed according to the method of Austin & Benet [10]. Quantification of 3FL and LNFP-II was performed against genuine standards purchased from Elicity Crolles, France. All other HMOs were quantified against maltotriose Sigma, Buchs, Switzerland as a surrogate standard assuming equimolar response factors

### Polygenic Score to Predict High or Low Abundance of 3FL in Milk of Expressor Mothers

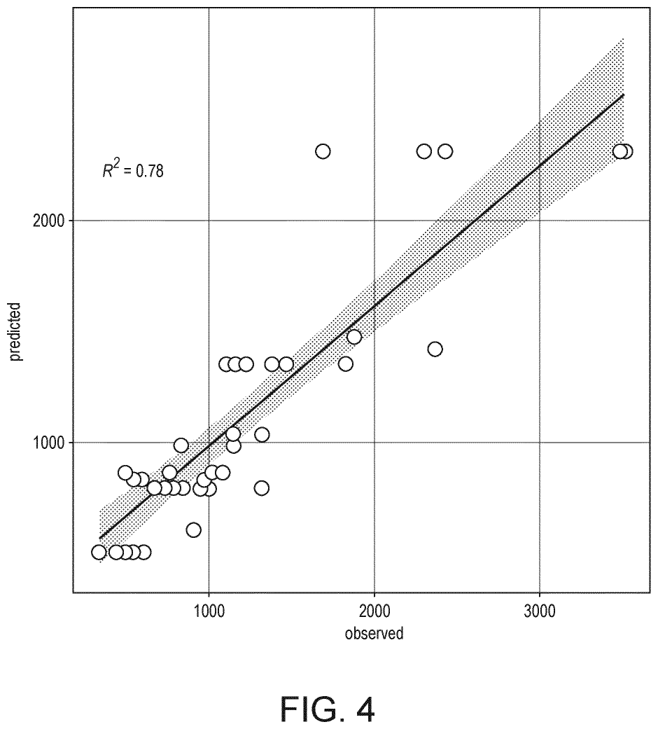

The selection of SNPs is performed by stepwise-selection of Generalized Linear Model (GLMs) in both directions. The selected model has been trained on a training dataset 300 repeats of 40-Fold CV. The evaluation of the model was performed on an independent test dataset. The selected SNPs were included in an algorithm to compute a genetic score. The genetic score was defined as the sum of the alleles for the SNPs selected in the model. Then we regressed the 3FL concentration with the genetic score on a training set to define the prediction model. Eventually we tested the prediction on an independent test dataset. We showed the genetic score is able to predict the levels on 3FL with an adjusted R Square to 0.78 (see Table 4 and FIG. 4).

TABLE 4
Summary table for the SNP included in the genetic score.

| | | Standard | |
|---|---|---|---|
| SNP | Coefficient | Error | P value |
| rs516316 | 0.54808 | 0.04813 | <0.001 |
| rs812936 | −0.43406 | 0.07127 | <0.001 |
| rs3894326 | −0.5902 | 0.10153 | <0.001 |
| rs1800022 | 0.66273 | 0.26056 | 0.0122 |

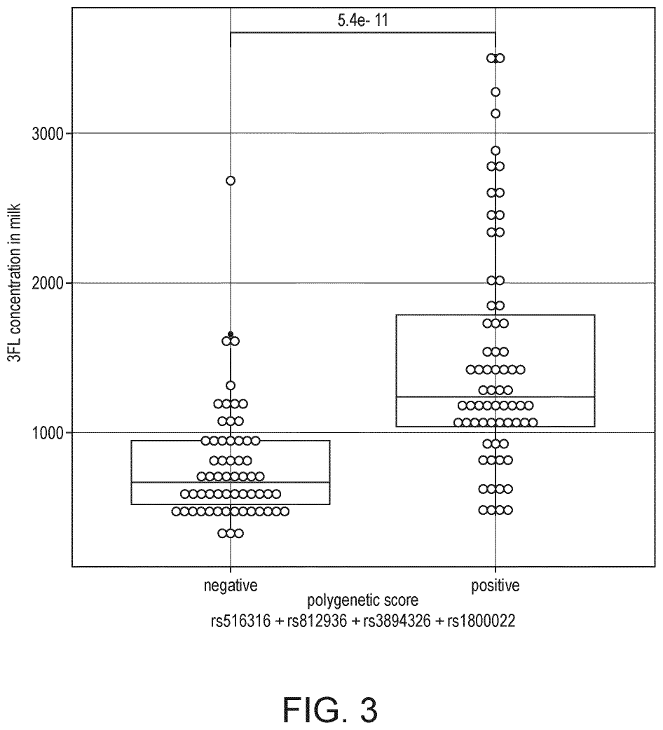

It was thus shown that expressor population could be divided into two sub populations with moderate and high levels of 3FL. The polygenic model proposed could justly predict the 3FL levels secreted in mothers. A zero or negative score predicts a moderate 3FL level, while a positive score predicts a high amount of 3FL (FIG. 3 and FIG. 4). Though 3FL levels are mainly associated with rs812936 polymorphism and heterozygous mothers are predominantly represented in the moderate level group, our polygenic score significantly outperforms predictions based on rs812936 alone p<0.001.

### Example 4

### Prediction of Levels of 3FL which Will be Secreted by a Pregnant Woman at 3 Months' Time of Lactation

DNA extracted from a saliva sample of a pregnant woman is genotyped for the following SNPs: rs516316, rs812936, rs3894326, and rs1800022. The following model is applied to obtain a polygenic score for the woman: ps=[f (rs516316)]×0.54808+[f (rs812936)]×−0.43406+[f(rs3894326)×−0.59029]+[f (rs1800022)]×0.66273, wherein factor f is attributed for each SNP as follows: the factor (fMM) is equal to 0 for all SNPs having two major alleles, the factor (fMm) is equal to 1 for all SNPs resulting to have one major and one minor allele and the factor (fmm) being equal 2 for all genes having 2 minor alleles.

If the polygenic score obtained is positive, then the woman is predicted to secrete levels of 3FL equal or higher than 890 mg/L at three months of lactation; If the polygenic score obtained is negative, then the woman is predicted to express levels of 3FL lower than 890 mg/L at three months of lactation.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. (canceled)

2. Method for attributing a polygenic score (ps) to a human subject, the method comprising:
a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);
b) Determining that the human subject belongs to the group of Non-secretors if SNP is calculated as follows:
ps=[f(rs601338)]×−0.56304+[f(rs28362459)]×0.39504+[f(rs778986)×0.18314+[f(rs1800022)]×−0.36211+[f(rs281377)]×0.10313
wherein f represents a factor [f (SNP)] attributed to each listed SNP (single nucleotide polymorphism), the factor (fMM) being equal to 0 for all SNPs having two major alleles (MM), the factor (fMm) being equal to 1 for all SNPs having one major and one minor allele (Mm) and the factor (fmm) being equal 2 for all SNP having two minor alleles (mm) rs601338 has two minor alleles (mm) or if SNP rs601338 has two major alleles (MM) or one major and one minor allele (Mm) determining that the subject belongs to the group of Secretors and continuing with step c);
c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject; and
d) calculating the polygenic score (ps) for the subject by using the following equation:
ps=[f(rs601338)]×−0.56304+[f(rs28362459)]×0.39504+[f(rs778986)×0.18314+[f(rs1800022)]×−0.36211+[f(rs281377)]×0.10313.

3. The method according to claim 2 which is performed in combination with a method for attributing a polygenic score for predicting levels of alpha-1,3/4-fucosylated human milk oligosaccharides expressed in breast milk by a human subject during lactation, wherein such method for method for attributing a polygenic score for predicting levels of alpha-1,3/4-fucosylated human milk oligosaccharides comprises:
Genotyping a human subject for the following SNPs: rs516316, rs812936, rs3894326, and rs1800022 and determining whether the human subject carries for each of such SNPs two major alleles (MM), one 20 major and one minor allele (Mm) or two minor alleles (mm);
Determining that the human subject belongs to the group of non expressors if any of the SNPs rs3745635, rs28362459, rs3894326, rs812936 has two minor alleles (mm) or if SNP rs601338 has two major alleles (MM) or one major and one minor allele (Mm) determining that the subject belongs to the group of expressors and continuing with step c);
attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject; and
calculating the polygenic score (ps) for the subject by using the following equation:
ps=[f(rs516316)]×0.54808+[f(rs812936)]×−0.43406+[f(rs3894326)×−0.59029]+[f(rs1800022)]×0.66273.

4. Method for predicting levels of alpha-1,2-fucosylated human milk oligosaccharides secreted in breast milk by a human subject during lactation based on a polygenic score calculated as follows:
ps=[f(rs601338)]×−0.56304+[f(rs28362459)]×0.39504+[f(rs778986)×0.18314+[f(rs1800022)]×−0.36211+[f(rs281377)]×0.10313
wherein f represents a factor [f (SNP)] attributed to each listed SNP (single nucleotide polymorphism), the factor (fMM) being equal to 0 for all SNPs having two major alleles (MM), the factor (fMm) being equal to 1 for all SNPs having one major and one minor allele (Mm) and the factor (fmm) being equal 2 for all SNP having two minor alleles (mm), the method comprising:
a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);
b) Determining that the human subject belongs to the group of Non-secretors if SNP rs601338 has two minor alleles (mm) or if SNP rs601338 has two major alleles (MM) or one major and one minor allele (Mm) determining that the subject belongs to the group of Secretors and continuing with step c);
c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;
d) calculating the polygenic score (ps) for the subject by using the following equation:
ps=[f(rs601338)]×−0.56304+[f(rs28362459)]×0.39504+[f(rs778986)×0.18314+[f(rs1800022)]×−0.36211+[f(rs281377)]×0.10313; and
e) if the polygenic score resulting from step d) is negative or zero, determining that the human subject belongs to the group of moderate secretors, while if the polygenic score resulting from step d) is positive, determining that the human subject belongs to the group of high secretors.

5. A method according to claim 4 which is performed in combination with a method for predicting levels of alpha-1,3/4-fucosylated human milk oligosaccharides secreted in breast milk by a human subject during lactation, wherein such method for predicting levels of alpha-1,3/4-fucosylated human milk oligosaccharides comprises:
Genotyping a human subject for the following SNPs: s516316, rs812936, rs3894326, and rs1800022 and determining whether the human subject carries for each of such SNPs two major alleles (MM), one major and one minor allele (Mm) or two minor alleles (mm);
Determining that the human subject belongs to the group of non expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);
attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;
calculating the polygenic score (ps) for the subject by using the following equation:
ps[f(rs516316)]×0.54808+[f(rs812936)]×−0.43406+[f(rs3894326)×−0.59029]+[f(rs1800022)]×0.66273; and
if the polygenic score resulting from step d) is negative or zero, determining that the human subject belongs to the group of moderate expressors, while if the polygenic score resulting from step d) is positive, determining that the human subject belongs to the group of high expressors.

6. Method for providing a nutritional recommendation for supplementation of alpha-1,2-fucosylated human milk oligosaccharides in the diet of an infant which is breast fed by a human subject, the method comprising:
a) Genotyping a human subject for the following SNPs: rs601338, rs28362459, rs778986, rs1800022 and rs281377 and determining whether the human subject carries for each of such SNPs two major alleles (MM), one major and one minor allele (Mm) or two minor alleles (mm);
b) Determining that the human subject belongs to the group of Non-secretors if SNP rs601338 has two minor alleles (mm) or if SNP rs601338 has two major alleles (MM) or one major and one minor allele (Mm) determining that the subject belongs to the group of Secretors and continuing with step c);
c) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;
d) calculating the polygenic score (ps) for the subject by using the following equation:
ps=[f(rs601338)]×−0.56304+[f(rs28362459)]×0.39504+[f(rs778986)×0.18314+[f(rs1800022)]×−0.36211+[f(rs281377)]×0.10313;
e) if the polygenic score resulting from step d) is negative or zero, determining that the human subject belongs to the group of moderate Secretors, while if the polygenic score resulting from step d) is positive, determining that the human subject belongs to the group of high Secretors; and
f) depending on which group the human subject belongs to providing the following nutritional recommendation:
f′) if the human subject belongs to the group of Non-secretors, recommending that the infant consumes synthetic nutritional compositions providing a total daily intake of 2′FL ranging from an amount of 1.2 g to 3 g; or
f″) if the human subject belongs to the group of moderate Secretors, recommending that the infant consumes synthetic nutritional compositions providing a total daily intake of 2′FL ranging from an amount of 0.8 g to 1.5 g; or
f″′) if the human subject belongs to the group of high Secretors and the infant is breast fed only partially, recommending that the infant consumes synthetic nutritional compositions providing a total daily intake of 2′FL ranging from an amount of 0.8 g to 1.5 g.

7. A method according to claim 6 for providing a nutritional recommendation for supplementation of alpha-1,2-fucosylated human milk oligosaccharides and of alpha-1,3/4-fucosylated human milk oligosaccharides in the diet of an infant which is breast fed by a human subject which also comprises:
A′) Genotyping a human subject for the following SNPs: rs516316, rs812936, rs3894326, and rs1800022 and determining whether the human subject carries for each of such SNPs two major alleles (MM), one major and one minor allele (Mm) or two minor alleles (mm);
B′) Determining that the human subject belongs to the group of non expressors if any of the SNPs rs3745635, rs28362459, rs3894326, rs812936 has two minor alleles (mm) or if any of the SNPs rs3745635, rs28362459, rs3894326, rs812936 has two major alleles (MM) or one major and one minor allele (Mm) determining that the subject belongs to the group of expressors and continuing with step c);
C′) attributing a factor [f (SNP)] to all SNPs, the factor (fMM) being equal to 0 for all SNPs resulting to be MM, the factor (fMm) being equal to 1 for all SNPs resulting to be Mm and the factor (fmm) being equal 2 for all genes resulting to be mm from step a) for the human subject;
D′) calculating the polygenic score (ps) for the subject by using the following equation:
ps=[f(rs516316)]×0.54808+[f(rs812936)]×−0.43406+[f(rs3894326)×−0.59029]+[f(rs1800022)]×0.66273;
E′) if the polygenic score resulting from step d) is negative or zero, determining that the human subject belongs to the group of moderate expressors, while if the polygenic score resulting from step d) is positive, determining that the human subject belongs to the group of high expressors;
F′) depending on which group the human subject belongs to providing the following nutritional recommendation:
J′) if the human subject belongs to the group of non expressors, recommending that the infant consumes synthetic nutritional compositions providing a total daily intake of 3FL ranging from an amount of 1 g to 3 g; or
J″) if the human subject belongs to the group of moderate expressors, recommending that the infant consumes synthetic nutritional compositions providing a total daily intake of 3FL ranging from an amount of 0.5 g to 2 g; or
J″′) if the human subject belongs to the group of high expressors and the infant is breast fed only partially, recommending that the infant consumes synthetic nutritional compositions providing a total daily intake of 3FL ranging from an amount of 0.5 g to 2 g.

8. A method according to claim 7 which is implemented on computer means.

9. A method according to claim 6 wherein the alpha-1,2-fucosylated human milk oligosaccharides is 2′FL.

10. A method according to claim 6 wherein the alpha-1,3/4-fucosylated human milk oligosaccharides is 3FL.

11. Method according to claim 6 wherein the human subject is a woman.

12-14. (canceled)
